(19) United States
(12) Patent Application Publication
(10) Pub. No.: US 2008/0027324 A1
(43) Pub. Date: Jan. 31, 2008

(54) Device for generation of a triggering signal

(76) Inventors: Helena Trojanova, Praha (CZ); Milos Bilwachs, Praha (CZ); Otto Lang, Plibram (CZ)

Correspondence Address: Hovey Williams LLP, Suite 400, 2405 Grand Blvd., Kansas City, MO, 64108, US

(21) Appl. No.: 11/496,595

(22) Filed: Jul. 31, 2006

(51) Int. Cl. A61B 8/00

(52) U.S. Cl. 600/454

(57) ABSTRACT

A device for generation of a triggering signal (8) for synchronized acquisition of heart images in nuclear medicine, cardiology and radiology, comprising a detector connected to a circuit (6) of signal correction and an evaluation circuit (3). The detector includes a detector of physiological signals of arteries.

### TECHNICAL FIELD

This invention concerns a device for generation of a triggering signal for a gated (synchronized) acquisition of heart images in nuclear medicine, cardiology and radiology, comprising a detector connected to the signal-correcting circuit and the evaluation circuit.

### BACKGROUND ART

Cardiac evaluations by the imaging method are EKG-gated to be able to record and compare images in the same phases of the heart cycle. In nuclear medicine (with Single Photon Emission Computer Tomography SPECT and with Positron Emission Tomography PET) and in radiology (with Computer Tomography CT and with Magnetic Resonance Imaging MRI) the recording of data for cardiac evaluation lasts a relatively long time (minutes). The proper total evaluation is, therefore, divided into a number of evaluations, each of which corresponds to one phase of heart cycle and then synchronized in order to ensure a recording in the correct time interval. An ultrasound cardiac evaluation has a high time resolution (microseconds), but the probes allow recording of only one cross-section through the organ. The synchronization by a triggering signal is, therefore, necessary in order to facilitate a comparison of different tomographic cross-sections of the heart in the individual heart phases.

The evaluation synchronized by the EKG triggering signal allows one to assess and quantify different parameters of global and regional function of the left heart ventricle; it, therefore, improves diagnostic possibilities and a prognosis. Above all, it allows for the creation of a realistic 4-dimensional model (3 space dimensions plus time) of the movements of the left heart ventricle.

The EKG-gated synchronization is accomplished by the R wave (high electrical impulse that causes the contractions of heart ventricles) triggering and recording of the heart cycle in the computer; then the next R wave stops the recording of this cycle and, simultaneously, it starts the recording of the next cycle. During the heart cycle the images of individual heart phases are recorded in corresponding time intervals.

In some cases, however, the EKG triggering signal is not suitable for the synchronization, where its use could cause artifacts and misdiagnosis. Mainly, this is in the case of patients with pacemakers implanted for the support of heart activity. A pacemaker generates an electrical impulse similar to R wave that causes the contractions of heart ventricles. Depending on type of stimulation, EKG signal could, therefore, show one or two very similar impulses without a possibility to establish the correct time relations between them and the beginning of the heart cycle (i.e. start of heart contractions). Also, the stimulation during the examination could change depending on the patient's momentary condition.

For these reasons it is necessary to use another signal that would have a firmly defined timing relation to the heart ventricle movements for the synchronization of acquisition in connection with the cardiac examination of patients with pacemakers, on which it would be possible to easily and unambiguously detect a certain periodically (in the rhythm of heart cycle) appearing point.

For the diagnostics, it is very important to have correct images of End-diastole (the maximum fill of ventricles) and End-systole (the minimum fill of ventricles). During the recording of individual images of a heart cycle the several last images of the cycle get distorted due to the uneven length of heart cycles. It is, therefore, necessary, in order to avoid the distortion of End-diastole and End-systole images, to have the triggering signal between diastole and systole.

### SUMMARY OF THE INVENTION

The above-mentioned problem is solved by a device for generation of the triggering signal for synchronized acquisition of heart images in nuclear medicine, cardiology and radiology, in accordance comprising a detector connected to the signal correction circuit and the evaluation circuit, in accordance with the invention, wherein the detector comprises a detector of physiological signals from arteries.

The generation of a triggering signal based on a recording of physiological signals from arteries has the advantage as compared with the EKG gated signals in that it is not negatively influenced by the pacemaker activity or other disturbing effects (e.g. obese patient with low R-wave).

According to one advantageous implementation, the detector of physiological signals comprises of a detector of blood volume changes in arteries, and the evaluation circuit includes a block for establishment of the bottom point of saw tooth curve of arterial blood volume in relation to time.

By measuring the volume of arteries into the evaluation circuit the arterial volume enters as a physiological variable. The volume of arterial blood is expressed by the saw tooth curve, which is an ideal controlling function enabling one to substantially exactly time the unblocking of the detector of the bottom turning point and a generation of a triggering signal.

The detector of blood volume changes in arteries could comprise an optical detector, with advantage of red LED diode and a corresponding photodiode for measuring of the passed through light or, respectively, reflected light; or a pneumatic detector of volume changes in upper/lower limb of a patient; or a capacity detector of volume changes of patient's body.

According to another advantageous implementation, the physiological signal detector comprises of a detector of blood flow speed in arteries, and the evaluation circuit comprises of a block for establishing of the maximum blood flow speed in arteries.

The detector of blood flow speed in arteries could comprise of an ultrasound Doppler probe.

### BRIEF DESCRIPTION OF THE DRAWINGS

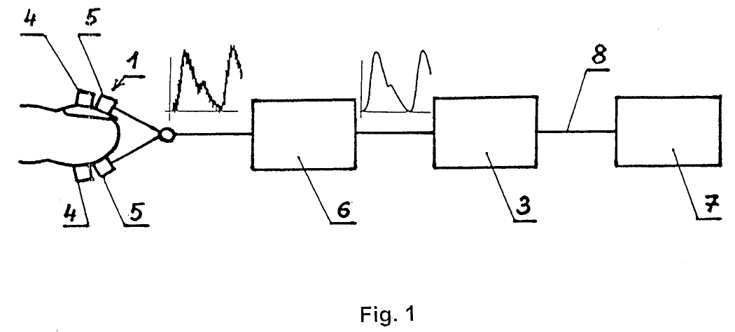

The invention is closer explained with help of drawings, where, on FIG. 1, there is schematically shown a device for generation of triggering signal with a detector of blood volume changes in arteries.

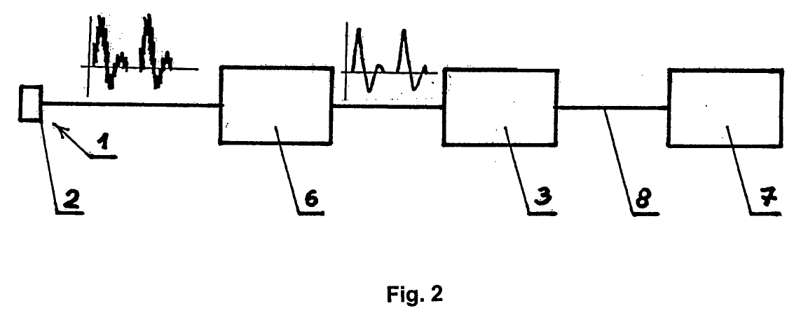

The FIG. 2 schematically shows a device for generation of a triggering signal with a detector of blood flow speed changes in arteries.

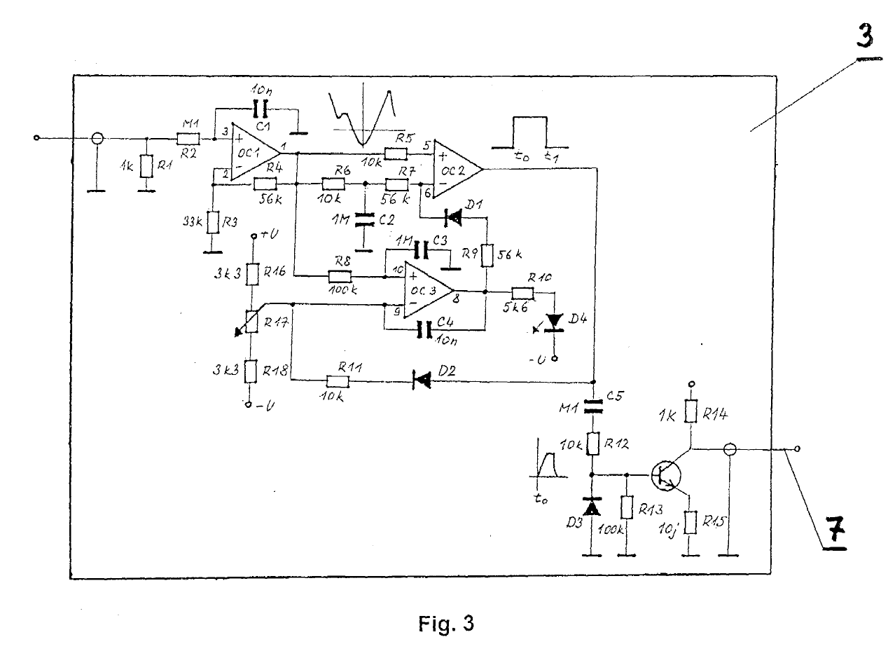

On FIG. 3 there is an example of a specific design of an evaluation circuit for a detection of the bottom point of the saw tooth curve of blood volume in arteries. According to this disclosure it will be obvious to those skilled in the art that the same function could be achieved by a number of differently designed electronic circuits generally intended for a detection of the bottom point of the curves.

### MODES FOR CARRYING OUT THE INVENTION

An example of realization of the device for generation of the triggering signal 8 for acquisition of heart images in nuclear medicine, cardiology and radiology, according to FIG. 1, comprises a detector of physiological signals of arteries, connected to circuit 6 for signal corrections and evaluation circuit.

As a first variant of the solution according to the invention, the detector of physiological signals comprises of detector 1 of blood volume changes in arteries. Those skilled in the art know of a number of specific realizations of such detectors. On FIG. 1, there is a schematically shown optical detector that includes red LED diode 4 and the corresponding photodiode 5 for measuring of passed-through, or respectively reflected light.

Further it is possible to use, as a detector 1 of blood volume changes in arteries, for example, a pneumatic detector of volume changes in an upper or lower limb of a patient, or a capacity detector of changes of a patient's body volume.

The evaluation circuit 3 with this first variant includes a block for detection of the bottom point of the saw tooth curve of the relation of blood volume in arteries with time. A specific example of design of evaluation circuit 3 is shown on FIG. 3 and includes an input filter OC1, a detection of the lowest point block OC2, a blocking circuit OC3, and a block of blocking threshold adjustment R17.

The first variant of solution according to the invention, based on a principle of measuring changes of blood volume in arteries, derives from a known fact that, by ejection of blood from the left ventricle into the aorta, the elastic wall of the aorta expands and this pulse (pressure) wave spreads through the aorta and its branches, and it is palpable as an arterial pulse. The pulse wave expatiates independently of the arterial course speed. The speed of expansion is approximately 5-8 m/s. The changes generated by the-pulse wave in the arterial wall are measured, according,to FIG. 1, by an optical detector that includes the red LED diode 4 and the corresponding photodiode 5.

With the blood pressure changes in connection with a periodic heart activity, the capillary capacity is changed and causes a change of absorption, reflection and diffusion of the light. Light pulses are emitted from the red LED diode 4 with constant intensity and the measurement of the passed-through, or respectively, reflected light is indirectly proportional to the blood volume in a tissue (i.e. arterial volume). The red LED diode 4 generates light from the red part of spectrum with advantage of a wave length from 640 nm up to 1000 nm. The amount of passed-through, or respectively, reflected light is measured by the photodiode 5. In order to obtain the least possible delay of a volume change of the measured arteries against the heart pulsation, it is recommended to attach the detector 1 of the volume changes to a finger, forehead, or ear of the patient.

The form of uncorrected oscillation course of the blood volume in arteries relation with time as the output from the detector 1 of blood volume changes is schematically shown on FIG. 1.

This signal is brought to the circuit 6 of signal correction, where it is amplified and the noise is filtered out by a known process. A number of specific designs of such circuits are well-known to those skilled in the art.

The course of the signal after correction is also schematically shown on FIG. 1. The shape of the curve of the arterial blood volume relation to time has a saw tooth course. The ascending side of the curve corresponds to the quick expansion of artery upon the contraction of heart muscle; the descending side corresponds to a slow shrinking of the artery during the heart chamber filling. The shape of the curve varies according to the condition of the heart and arterial walls. The shape of the curve is primarily influenced by the heart function, its ability to contract and eject the blood from ventricles. Secondarily, the shape of the curve is influenced by the arterial walls. The viscous traits of arterial walls are very important not only for the speed of pulse wave expansion through the vascular system, but also for the attenuation of its different harmonic components. It is why, according to the condition of the heart and arteries, the curves of the arterial volume changes in different patients have different shapes. With some patients, roughly in the middle of the descent between maximal and minimal value of the curve, there appears an indication of a plateau, or even a mild increase, at the time of aortal valve closing; with other patients this plateau is less pronounced.

The corrected signal from the circuit 6 of signal correction is brought into the evaluation circuit 3, which detects the bottom point of the saw tooth curve of the blood volume in arterial channel relation to time, and generates the triggering signal 8. The evaluation circuit 3 must be sufficiently resistant against physiological deviations from the non-descending course of the descending side of the curve at the time of aortal valve closing. The solution is an adaptive circuit deriving the sensitivity of detection of the bottom point from the level in previous periods. The range of adaptation should be primarily adjustable. An ideal setting can in exceptional cases differ, but it does not have a fundamental effect on the evaluation results.

A short time difference of the triggering signal 8 from the diagnostically decisive phases of the heart activity (End diastole) defines more precisely the heart cycle phasing, reduces a statistical variation of the result, and gives sharper images, and more precise results. Those are beneficial reasons for generation of the triggering signal 8 at the bottom point of the saw tooth curve.

With a realization that is not shown, it is further possible to measure even the period of a heart cycle and, for the generating of a triggering signal 8, to use a weighted influence of both quantities, i.e. both, the time of period of previous heart cycles and the volume of arteries, and to enlarge the adaptive circuit for detection of the bottom point with a detection window opened according to the measured period of the heart cycle. This feature makes the described device more precise and more resistant against disturbance than the currently used EKG.

As a basis for the device for generation of the triggering signal 8 for acquisition of heart images in nuclear medicine, cardiology and radiology, it is possible to use a well-known oximeter, supplemented by an evaluation circuit 3 that includes a block for establishing the bottom point of the saw tooth curve of the blood volume in arteries relation to time.

The generated triggering signal 8 can then be used in nuclear medicine, cardiology and radiology for synchronization of a heart image recording in any medical imaging device 7, for example, in a Single Photon Emission Computer Tomography SPECT, Positron Emission Tomography PET, Computer Tomography CT, Magnetic Resonance Imaging MRI, and ultrasound examination.

In a second variant of solution according to the invention, the physiological signal detector comprises of a detector 2 of blood flow speed changes in arteries. Those skilled in the art know a number of specific designs of such detectors. On FIG. 2, there is schematically drawn an ultrasound Doppler probe.

The evaluation circuit 3 in this design includes a block for establishing the maximum speed of blood flow in arteries. One skilled in the art is able to design, without further explanation or undue experimentation, a number of specific connections of such circuit.

The device according to the invention benefits from the fact that the blood flow speed in arteries changes during the heart cycle; the maximum speed is achieved at the beginning of the left ventricle contraction.

The curve of the blood flow speed in arteries has one pronounced peak at the moment of emptying the ventricles with the heart muscle contraction, and another, smaller one, after the heart valve closing. An ideal point for generation of the triggering signal 8 is the moment of the maximum blood flow speed in the arteries, because this moment can be easily detected on the curve and is always between diastole and systole. This maximal value can slightly differ in various heart cycles; therefore, it is necessary to derive the moment for generation of the triggering signal 8 from the local maximum, and not from absolute value of blood flow speed.

The shape of an uncorrected saw-tooth curve of the blood flow speed in arteries in relation to time at the output from the detector 2 showing changes of the arterial blood flow speed is schematically outlined on FIG. 2.

This signal is brought to the circuit 6 of signal correction, where it is amplified and the noise is filtered out by known methods. There are several specific designs of such circuits known to those skilled in the art.

The corrected signal is brought from the circuit 6 of signal correction to the evaluation circuit 3, which detects the maximum in the curve of blood flow speed course in arteries and generates the triggering signal 8.

The evaluation circuit 3 must be sufficiently resistant against physiological deviations in absolute values of blood flow speed during the heart cycles. A solution is an adaptive circuit deriving the sensitivity of the maximum detection from the level in previous periods. Those skilled in the art know a number of specific designs of such circuits; one such circuit used for the detection of the bottom point of a saw tooth curve is shown on FIG. 3. The range of adaptation should be primarily adjustable. An ideal setting can differ in exceptional cases, but it does not fundamentally affect the results of examination.

The generated triggering signal 8 is then used, as described above, in nuclear medicine, cardiology and radiology for triggering of acquisition of heart imagingon any medical imaging device 7 to which the circuit 3 is connected as shown in FIG. 2.

### LIST OF REFERENCE MARKS

1 detector of blood volume changes in arteries

2 detector of blood flow speed in arteries

3 evaluation circuit

4 red LED diode

5 photodiode

6 circuit of signal correction

7 medical imaging device

8 triggering signal

1. A device for generation of a triggering signal for synchronized acquisition of heart images in nuclear medicine, cardiology and radiology, comprising:
a circuit of signal connection;
an evaluation circuit; and
a detector connected to the circuit of signal correction and to the evaluation circuit, wherein the detector comprises a detector of physiological signals of arteries.

2. The device according to claim 1, wherein the detector of physiological signals comprises of a detector of blood volume changes in arteries, and the evaluation circuit includes a block for detecting the bottom point of a saw-tooth curve of relation of blood volume in arteries to time.

3. The device according to claim 2, wherein the detector of blood volume changes in arteries comprises an optical detector.

4. The device according to claim 3, wherein the optical detector includes a red light emitting diode and a photodiode for measuring light.

5. The device according to claim 4, wherein the light measured by the photodiode includes both passed-through light and reflected light.

6. The device according to claim 2, wherein the detector of blood volume changes in arteries comprises a pneumatic detector of volume changes in a limb of a patient.

7. The device according to claim 2, wherein the detector of blood volume changes in arteries comprises a capacity detector of volume changes in a body of a patient.

8. The device according to claim 1, wherein the detector of physiological signals comprises of a detector of the blood flow speed curve in arteries, and the evaluation circuit includes a block for detecting the maximum of blood flow speed in arteries.

9. The device according to claim 8, the detector of the blood flow speed curve in arterial channel comprises an ultrasound Doppler probe.
